(19) United States
(12) Patent Application Publication
(10) Pub. No.: US 2016/0151493 A1
(43) Pub. Date: Jun. 2, 2016

(54) Heating Elements Having Plasmonic Nanoparticles for Localized Heating

(71) Applicant: UNIVERSITY OF SOUTH FLORIDA, Tampa, FL (US)

(72) Inventor: Anna Pyayt, Tampa, FL (US)

(73) Assignee: UNIVERSITY OF SOUTH FLORIDA, Tampa, FL (US)

(21) Appl. No.: 14/778,703

(22) PCT Filed: Jun. 3, 2015
(86) PCT No.: PCT/US15/34003

§ 371 Date: Sep. 21, 2015

Related U.S. Application Data
(60) Provisional application No. 62/007,125, filed on Jun. 3, 2014.

(51) Int. Cl. A61K 41/00; A61K 47/48; A61K 9/00; A61N 5/06; A61B 18/04

(52) U.S. Cl. CPC A61K 41/0052; A61N 5/062; A61B 18/04; A61K 9/0019; A61K 47/4893; A61K 47/48015; A61K 41/00; A61N 2005/0659

(57) ABSTRACT

In one embodiment, a heating element includes a central particle and a plurality of plasmonic nanoparticles attached to the central particle, wherein the nanoparticles undergo plasmonic heating when exposed to near infrared light.

### CROSS-REFERENCE TO RELATED APPLICATION

This application claims priority to co-pending U.S. Provisional Application Ser. No. 62/007,125, filed Jun. 3, 2014, which is hereby incorporated by reference herein in its entirety.

### BACKGROUND

Inorganic nanocages are hollow nanoparticles that absorb light in the near-infrared (NIR) where biological tissues absorb the least amount of light. Because they are biocompatible, nanocages can be injected into a target object, such as a tumor, and can be heated through the application of NIR light to kill the surrounding cells.

While nanocages are useful in the above-described application, they cannot be controlled. Because they are so small, they can undesirably migrate to other organs of the body. In addition, their small size can make it difficult for the body to remove the nanocages from the body. It would be desirable to be able to use nanoparticles without these drawbacks.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1 is a scanning electron microscope (SEM) image of a single heating element. The inset image shows a magnified portion of the heating element and its plasmonic nanoparticles.

FIG. 2 is a SEM image of fabricated plasmonic nanocages prior to being attached to central particles to form the heating elements.

FIG. 3 is a schematic diagram of an experimental apparatus used to test the heating capabilities of the heating elements.

FIGS. 4A-4D are optical microscopy images of an elastin-like polypeptide (ELP) solution containing heating elements both before and after the heating elements are exposed to near infrared (NIR) light.

FIGS. 5A-5D are optical microscopy images of an ELP solution containing heating elements illustrating the amount of heat generation as a function of the number of heating elements exposed to NIR light.

FIGS. 6A-6C are optical microscopy images of an ELF solution containing heating elements illustrating the amount of heat generation as a function of the alignment between the heating elements and the NIR light.

FIGS. 7A-7D are optical microscopy images illustrating controlling heating elements by moving them across a Petri dish using an external magnet.

FIGS. 8A-8D are optical microscopy images illustrating destruction of cells using heating elements.

### DETAILED DESCRIPTION

As described above, it would be desirable to be able to use nanoparticles within the body without the drawbacks associated with free nanocages, including difficulty in controlling the nanocages after injection and removing the nanocages from the body, Disclosed herein are heating elements that comprise plasmonic nanoparticles that are attached to the outer surfaces of larger central particles. The nanoparticles can be used to provide localized heating like free nanocages but, because they are attached to the central particles, they will not migrate as easily and are easier for the body to remove. In some embodiments, the central particles are magnetic so as to enable manipulation of the heating elements to further control their location in the body.

In the following disclosure, various specific embodiments are described. It is to he understood that those embodiments are example implementations of the disclosed inventions and that alternative embodiments are possible. All such embodiments are intended to fall within the scope of this disclosure.

A large amount of research is being performed on gold nanocages that can be engineered to he extremely efficient nano-heaters operating at the wavelength least absorbed by tissue and thus used for photothermal cancer therapies. Their efficiency for the cancer treatment has been successfully demonstrated in animal models. One of the limitations of the use of such nanocages is that it is difficult to maintain them within specific areas of the body because they tend to migrate from the injection site and there is no mechanism for controlling their location once injected. In view of this, there is a need to control the position of the nanocages within the body. Disclosed herein is a solution in which heating elements comprising central particles and plasmonic nanoparticles are injected into the body, In some embodiments, the central particles are magnetic particles to which the nanoparticles are attached. In such cases, the nanoparticles of the heating elements can be used to provide plasmonic heating and the positions of the heating elements can be magnetically controlled from outside of the body with a magnet.

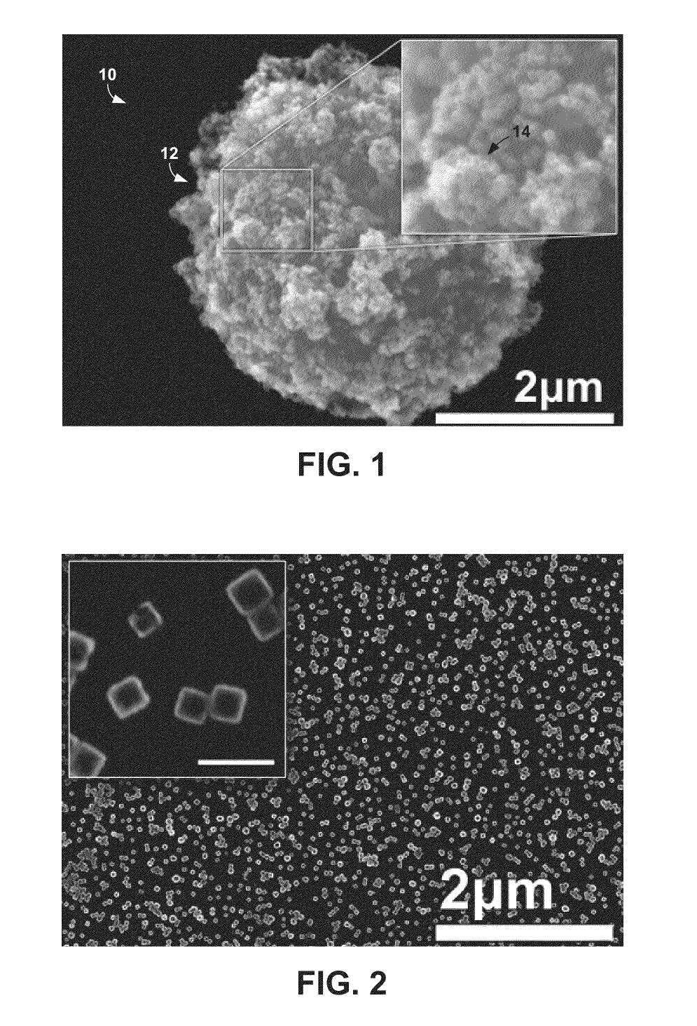

FIG. 1 is a scanning electron microscope (SEM) image of an example heating element 10 that can, for example, be injected into a target site within living tissue. Generally speaking, the heating elements 10 comprise a nano- or micro-sized central particle 12, in the form of a nano- or microbead, to which a plurality of plasmonic nanoparticles 14, such as nanocages, are attached. The central particles 12 can have an outer dimension (e.g., diameter) of approximately 100 nm to 10 μm. As noted above, the central particles 12 can be magnetic, in which case they can comprise a magnetic material, such as an iron-oxide material or chromium dioxide. In some embodiments, the magnetic material can be encapsulated in one or more other materials that facilitate attachment of the plasmonic nanoparticles. In some embodiments, the central particles have bumpy outer surfaces that increase the surface coverage of the nanoparticles.

The plasmonic nanoparticles 14 can comprise hollow, porous gold nanocages (e.g., cubic particles) having an outer dimension (e.g., width or height) of approximately 30 to 50 nm. For the remainder of the disclosure, the plasmonic nanoparticles 14 will be assumed to comprise such plasmonic nanocages. The nanocages absorb near-infrared (NIR) light (e.g., laser light at a wavelength of ˜808 nm) and undergo plasmonic heating that can be used to kill nearby cells, such as cancer cells.

Fabrication of the heating elements will now be described. The plasmonic nanocages can first be prepared using a galvanic replacement reaction between silver nanocubes and HAuCl₄. For example, 42 nm gold nanocages were prepared using a galvanic replacement reaction between 36 nm silver nanocubes and HAuCl₄ in an aqueous solution. The UV-visible (UV-Vis) spectra of these gold nanocages were monitored using a PerkinElmer Lambda 750 UV/vis/NIR spectrophotometer (Waltham, Mass.). The localized surface plasmon resonance (LSPR) peak was centered at 785 nm. The nanocages were purified by centrifugation at 10,000 rpm for 10 minutes and washed twice with water. The product was finally redispersed in water at 1.8 nM as the stock solution. FIG. 2 is a SEM image of the fabricated gold nanocages as deposited on a silicon substrate. The size of each individual nanocage was between 30 and 50 nm. The nanocages formed less than a monolayer of nanocages on the substrate surface.

Once the plasmonic nanocages have been fabricated, they can be attached to the outer surfaces of the central particles. In some embodiments, the magnetic material of the central particles can he encapsulated in a polymer, such as polystyrene, that is, in turn, coated with an amino layer that facilitates the attachment of the nanocages. In such cases, the plasmonic nanocages can be attached to the central particles through simple incubation at a temperature of approximately 23 to 25° C. and a time period of approximately 60 to 120 minutes. Alternatively, such attachment can be achieved by mixing the polymer-encapsulated central particles and the plasmonic nanocages in an amino solution. For example, 100 μl of 10% (v/v) of gold nanocage solution was mixed with magnetic microbeads extracted from 300 μl of 2.5% (w/v) AMS-40-10H amino-coated Sphero magnetic beads solution by isolating them using a magnet. The solution was thoroughly mixed and allowed interact for one hour at room temperature. After that all the nanocages that were not attached to the microbeads were washed away, while the magnetic beads were captured inside of the glass vial using an external magnet.

After the heating elements have been fabricated, they can be injected into living tissue, such as living human tissue, for the purpose of killing cells, such as tumor cells. In some embodiments, the heating elements can be suspended in a liquid, such as a biocompatible water-based liquid, to facilitate such injection.

The first property of the heating elements that must be demonstrated is that its nanocages retain their plasmonic properties so that the heating elements can operate as efficient heaters upon exposure to the MR light. In order to characterize theft heat generation properties, the heating elements were immersed in a solution of elastin-like polypeptides (ELPs) that changes its transmission to visible light depending on temperature. ELPs are derived from natural elastin and composed of repeated blocks of penta-peptide, Val-Pro-Gly-X-Gly, where X is a guest residue that can be any amino acid except Proline. ELPs undergo a phase transition with increases in temperature, self-assemble, and become opaque to visible light.

After immersion in an ELP solution, the heating elements can be exposed to MR radiation and the detailed heating profile can be observed under a microscope. While experimental visualization of plasmonic heating on the nano- and micro-scale is challenging using traditional approaches, this method enables repeated high-resolution thermal field imaging in real time without the need for expensive equipment or sophisticated chemical probes.

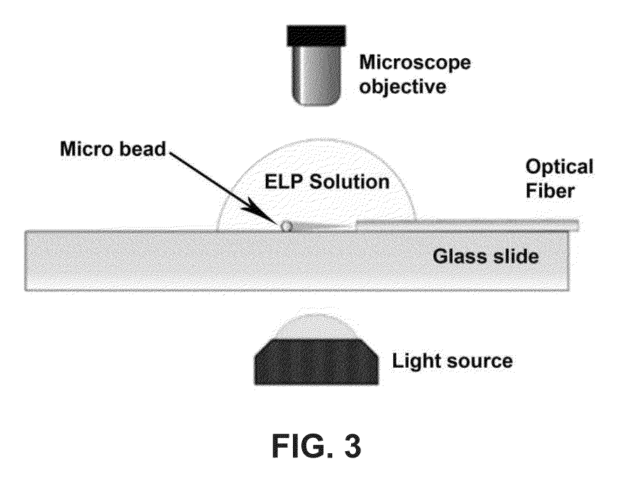

FIG. 3 illustrates an experimental apparatus that was used to perform heating experiments on the heating elements. As shown in this figure, a droplet of an ELF solution containing heating elements was deposited on a glass slide positioned beneath a microscope objective and above a light source. The heating elements were irradiated with NIR light emitted from an optical fiber. Images were taken using the microscope and an IR filter was used to filter out light shining out of the optical fiber. This way, changes in transparence of the ELP solution to visible light become very noticeable and the excitation light is not seen in the images.

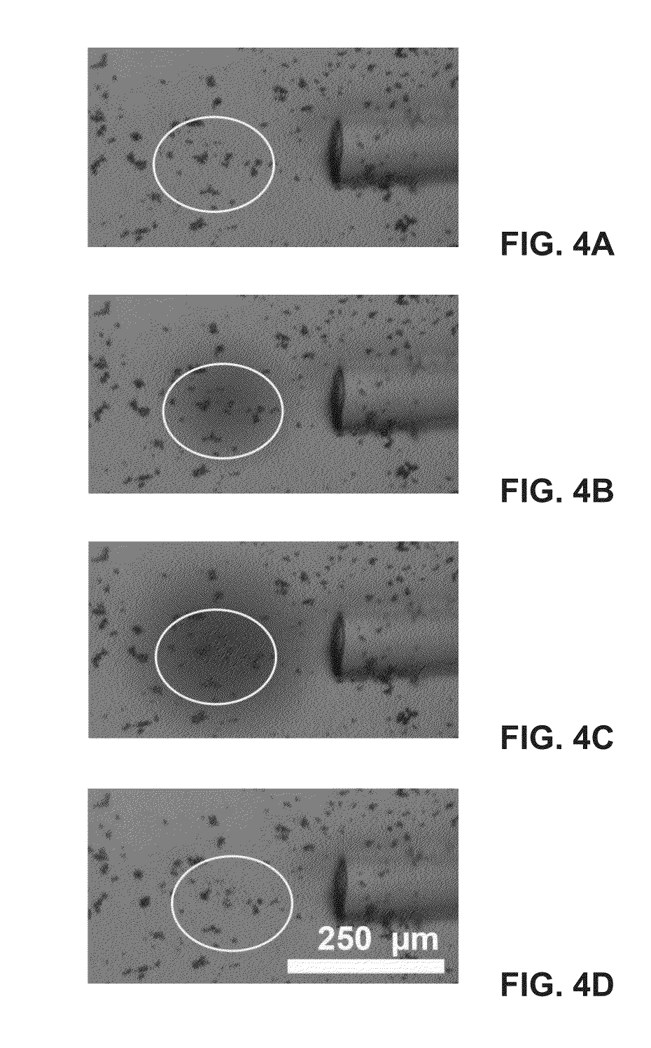

Example images that were captured are shown in FIGS. 4A-4D. In these figures, the white circle identifies the area in which the heating elements can be exposed to light from the optical fiber when the laser is turned on. Because the nanocages were only located on the surface of central particles, all of the healing occurred around the particles. FIG. 4A shows the ELP solution before the laser was turned on. FIG. 4B shows the ELP solution after the heating elements in the ELF solution were exposed to 30 mW laser light with a wavelength of 808 nm for 1 second. FIG. 4C shows the ELP solution after the heating elements in the ELP solution were exposed to the 30 mW laser light and reached a steady state approximately 5 seconds after the laser was turned on. FIG. 4D shows the ELP solution 5 seconds after the laser was turned off.

It can be appreciated from the images of FIG. 4 that heating of just a few heating elements in the center of the circle resulted in an increase in temperature in an area approximately 200 μm in diameter. The size and color of the dark area is determined by the heat balance between heating of the heating elements and heat transfer to the surrounding medium. The color of the solution can be directly correlated to a specific temperature. Heating of just several heating elements locally heated the solution above 30° C. (by more than 7° C.). This means that the individual plasmonic nanocages attached to the heating elements still have efficient heating properties due to exposure to NIR light. A steady state heating profile was reached after 5 to 15 seconds of exposure to light. After the laser was turned off, the heat quickly dissipated to the surrounding media and, within 5 seconds, the system returned to its initial state with the ELP solution being transparent, as shown in FIG. 4D.

There are several mechanisms that can be used to control heat generation and temperature increase in a particular area. A first mechanism is the laser power. By increasing the light intensity, the plasmonic nanocages and the surrounding medium can be heated to a higher temperature, However, for practical applications, such as photothermal cancer therapy, the maximum laser power increase is limited due to risk of damaging healthy tissue at high power levels.

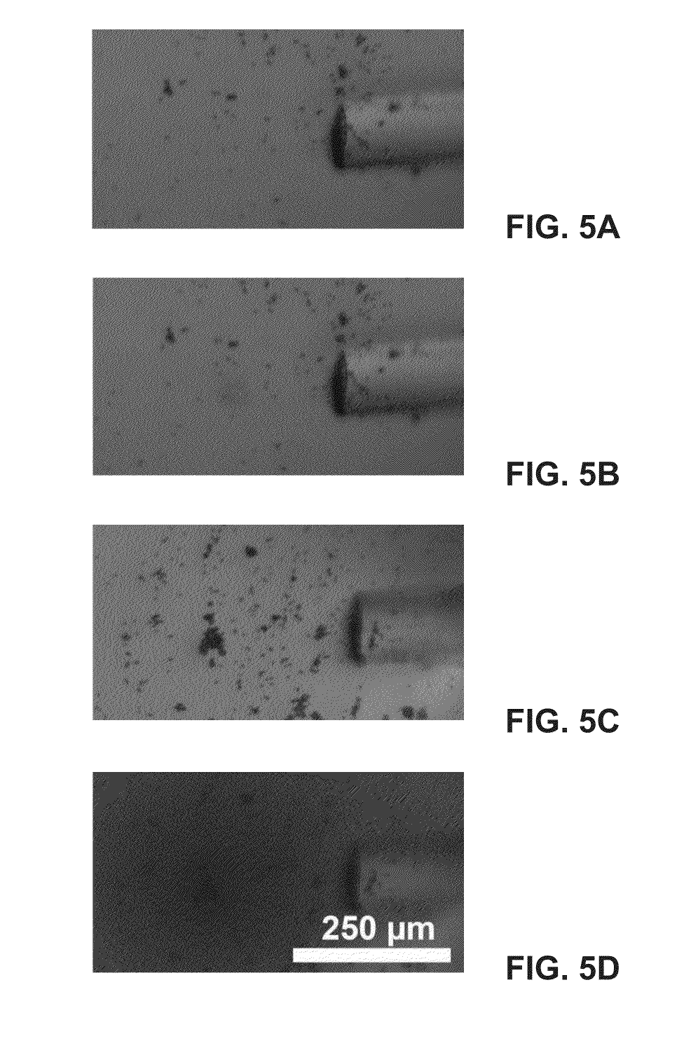

A second mechanism for controlling heat generation relates to controlling the volume of light-sensitive material exposed to the light radiation. FIGS. 5A-5D demonstrate that the amount of generated heat is directly related to the number of the heating elements exposed to NIR light. In FIGS. 5A and 5B, an ELP solution has a relatively low concentration of heating elements, while in FIGS. 5C and 5D, the ELP solution has a relatively high concentration of heating elements. FIGS. 5A and 5C show the solutions prior to the application of NIR light, while FIGS. 5B and 5D show the solutions after the application of NIR light at approximately 30 mW. When a single heating element was exposed to the light (FIG. 5B), there was a local increase of temperature around that element of approximately 3° C. When a clump of heating elements were exposed to the light (FIG. 5D), however, the temperature in the entire field of view increased from 23° C. to more than 30° C. and the area surrounding the clump increased in temperature to nearly 39° C. (an increase of more than 15° C.). This means that a local concentration of heating elements in a tumor can be used to significantly increase temperature in the tumor relative to the surrounding tissue during photothermal cancer therapy,

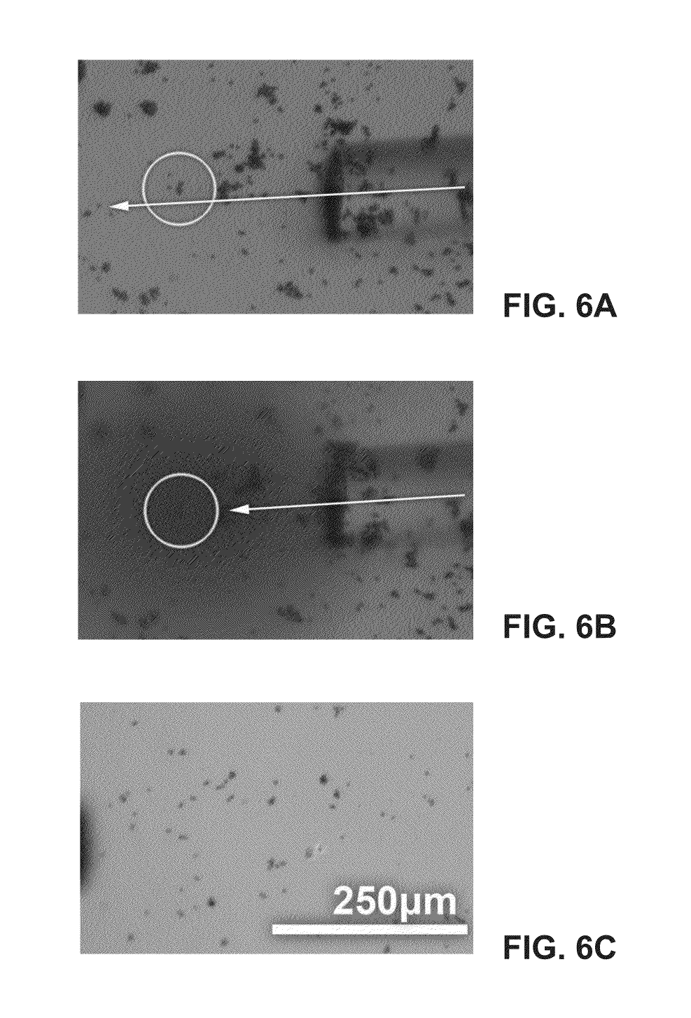

A third mechanism for controlling heat generation relates to controlling the alignment of the heating elements with the emitted light. FIGS. 6A and 6B demonstrate the sensitivity of heat generation versus the alignment of the heating elements with the laser radiation. In FIG. 6A, a small dump of heating elements is misaligned by just tens of micrometers with the center of the laser beam. Even though the laser was on, no heat was generated. In FIG. 6B, the optical fiber is aligned with the dump of heating elements and the area surrounding the clump was heated. This illustrates that precise alignment of the external light is very important during photothermal therapy. If the light is misaligned with the heating elements injected into a tumor, it could result in heating healthy tissue and not the tumor.

A control experiment was conducted in order to demonstrate that the heat being generated by the heating elements is related to localized surface plasmon resonance excitation of the nanocages and not due to the absorption of NIR by the central particles. FIG. 6C shows the results of this control experiment, in which central particles having no plasmonic nanocages are aligned with the optical fiber that emitted NIR light for 5 minutes. As is apparent in FIG. 6C, no heating was observed. This implies that the central particles do not significantly absorb or produce any visible heating effect due to NIR.

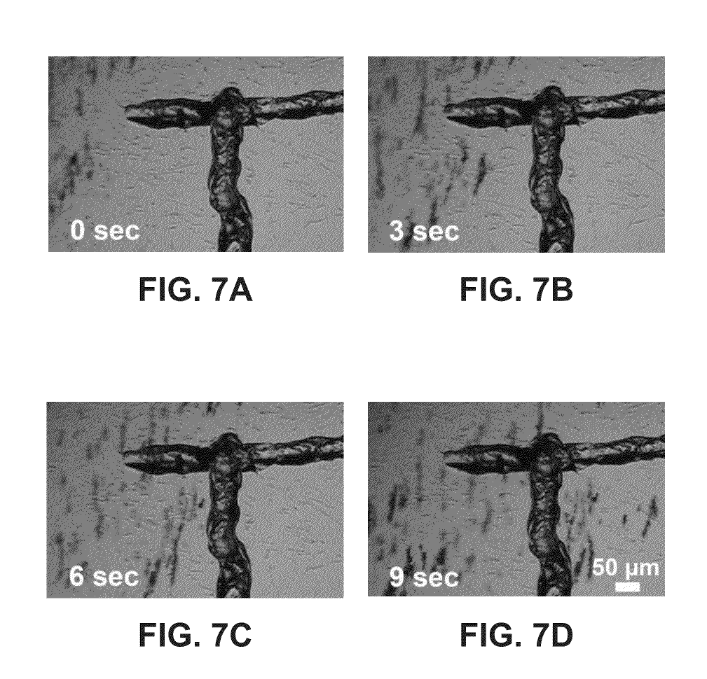

After demonstrating the efficient heating properties of the heating elements, the next step was to show that the heating elements also have efficient magnetic properties and can be controlled with an external magnet. FIGS. 7A-7D illustrate magnetic steering of the heating elements towards a “T” marker etched into the bottom of a Petri dish. Beginning with FIG. 7A, 1 μl of 1% (v/v) heating elements were drop-cast far from the “T” marker. Next, as illustrated in FIGS. 7B-7D, the heating elements were moved with an external magnet from the left side of the Petri dish to the right to the “T” marker. This shows that the heating elements can be magnetically delivered to any specific area of the Petri dish covered with cells, and then exposed to the NIR light, As a result, cells can be locally and controllably destroyed at a particular location.

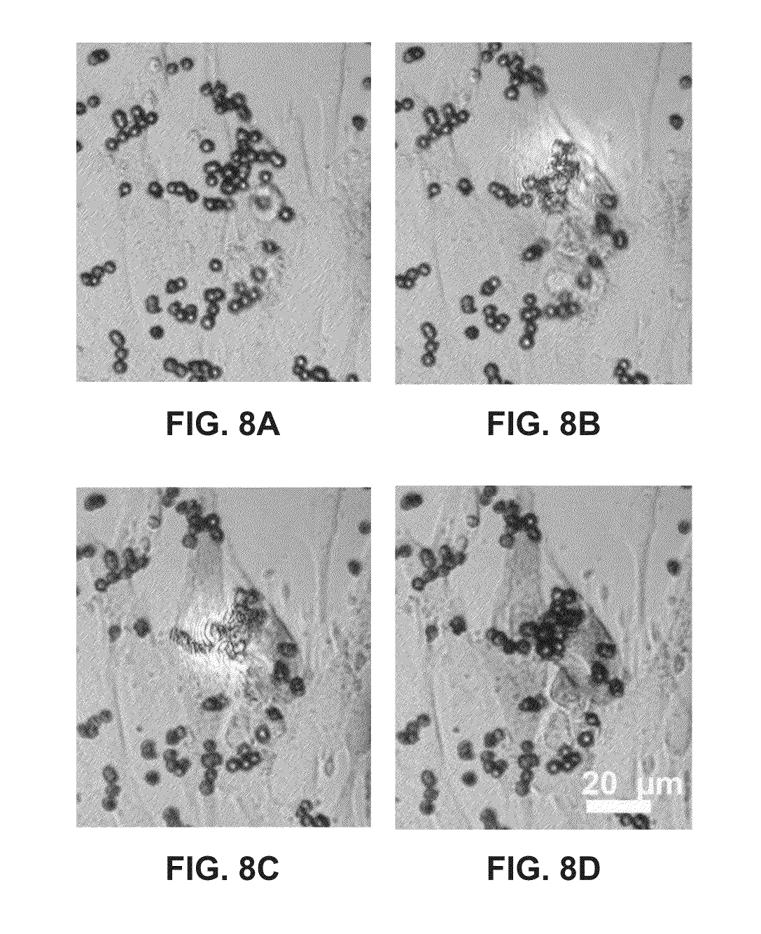

FIGS. 8A-8D illustrate controllable destruction of individual cells using a cluster of heating elements. The cells were live fibroblast cells. To distinguish between live and dead cells, 100 μl of Trypan blue solution was added to a solution in which the cells were immersed and allowed to mix with the cells for 3 minutes. Live fibroblast cells are transparent. When the cells die, however, they become blue due to diffusion of the Trypan blue into the dead cell. FIGS. 8A, 8B, 8C, and 8D show the cells after irradiation with NIR light after 0 minutes, 10 minutes, 20 minutes, and 25 minutes, respectively. As can be appreciated from these figures, the heating elements effectively killed nearby cells while neighboring cells survived. This demonstrates that the heating elements are effective in heating and photothermally killing cells.

1. A healing element comprising:
a central particle; and
a plurality of plasmonic nanoparticles attached to the central particle, wherein the nanoparticles undergo plasmonic heating when exposed to near infrared light.

2. The heating element of claim 1, wherein the central particle has a diameter of approximately 100 nanometers to 10 microns.

3. The heating element of claim 1, wherein the central particle is magnetic.

4. The heating element of claim 3, wherein the central particle is made of iron oxide-containing material.

5. The heating element of claim 1, wherein the plasmonic nanoparticles are hollow, porous nanocages.

6. The heating element of claim 5, wherein the nanocages have outer dimensions of approximately 30 to 50 nanometers.

7. The heating element of claim 5, wherein the nanocages are made of gold.

8. An injectable solution comprising:
a liquid; and
a plurality of heating elements contained in the liquid, the heating elements comprising central particles having plasmonic nanoparticles attached thereto, wherein the nanoparticles undergo plasmonic heating when exposed to near infrared light.

9. The solution of claim 8, wherein the liquid comprises a biocompatible water-based liquid.

10. The solution of claim 8, wherein the central particles have diameters of approximately 100 nanometers to 10 microns.

11. The solution of claim 8, wherein the central particles are magnetic.

12. The solution of claim 11, wherein the central particles are made of an iron oxide-containing material.

13. The solution of claim 8, wherein the plasmonic nanoparticles are hollow, porous nanocages.

14. The solution of claim 13, wherein the nanocages have outer dimensions of approximately 30 to 50 nanometers.

15. The solution of claim 13, wherein the nanocages are made of gold.

16. A method for killing cells within living tissue, the method comprising:
injecting heating elements into a target site within the living tissue, the heating elements comprising central particles to which a plurality of plasmonic nanoparticles are attached; and
exposing the heating elements to near infrared light to cause the plasmonic nanoparticles to rise to a temperature that kills adjacent cells within the living tissue.

17. The method of claim 16, wherein injecting the heating elements comprises injecting the heating elements into a tumor within living human tissue.

18. The method of claim 15, wherein the central particles have diameters of approximately 100 nanometers to 10 microns.

19. The method of claim 15, wherein the central particles are magnetic and further comprising controlling the positions the heating elements within the living tissue using a magnet positioned outside of the living tissue.

20. The solution of claim 19, wherein the central particles are made of an iron oxide-containing material.
